(19) United States
(12) Patent Application Publication
(10) Pub. No.: US 2025/0302277 A1
(43) Pub. Date: Oct. 2, 2025

(54) Endoscope with Voice Control

(71) Applicant: PSIP2 LLC, Manchester, NH (US)

(72) Inventors: John M. Cronk, Strafford, NH (US); Siddharth Desai, Mission Viejo, CA (US); Mark H. Getelman, Tarzana, CA (US); Bryan Lord, Bedford, NH (US); Nithin Shivashankar, Bangalore (IN)

(73) Assignee: PSIP2 LLC, Manchester, NH (US)

(21) Appl. No.: 19/091,432

(22) Filed: Mar. 26, 2025

Related U.S. Application Data
(60) Provisional application No. 63/570,678, filed on Mar. 27, 2024.
(60) Provisional application No. 63/662,337, filed on Jun. 20, 2024.
(60) Provisional application No. 63/666,540, filed on Jul. 1, 2024.
(60) Provisional application No. 63/739,458, filed on Dec. 27, 2024.
(60) Provisional application No. 63/770,259, filed on Mar. 11, 2025.

(51) Int. Cl. A61B 1/00; A61B 1/015; G06F 3/16; G10L 15/00

(52) U.S. Cl. CPC A61B 1/0004; A61B 1/00009; A61B 1/00066; A61B 1/015; G06F 3/167; G10L 15/00

(57) ABSTRACT

An endoscope with voice control. A data processor for the endoscope obtains voice training data for a specific surgeon, and/or information indicating spoken utterances associated with a surgical procedure for which the endoscope is to be used. Voice utterances signals are received from a surgeon during the surgery. Speech recognition technology decodes the voice utterance signals into commands to control the endoscope, based at least in part on the data obtained from the database, and control commands are issued to implement the decoded voice utterances. Concurrently with the accepting and decoding of voice utterances, the processor accepts and decodes control signals from at least one other input device. The processor issues control commands to components of the endoscope to implement the decoded other input control signals. The voice utterances are accepted, decoded, and executed concurrently with the other input control signals.

### BACKGROUND

This application claims benefit as a non-provisional of U.S. Provisional applications Ser. No. 63/770,259, filed Mar. 11, 2025, titled Endoscope With Voice Activation; U.S. Provisional Application Ser. No. 63/739,458, filed Dec. 27, 2024, titled Endoscope with Improved Fluid Flow; U.S. Provisional Application Ser. No. 63/666,540, filed Jul. 1, 2024, titled Endoscope; U.S. Provisional Application Ser. No. 63/662,337, filed Jun. 20, 2024, titled Endoscope; U.S. Provisional Application Ser. No. 63/570,678, filed Mar. 27, 2024, titled Endoscope with Voice Activation. The benefit applications are incorporated by reference.

This application relates to endoscopes, laparoscopes, arthroscopes, colonoscopes, and similar surgical devices or appliances specially adapted or intended to be used for evaluating, examining, measuring, monitoring, studying, or testing living or dead human and animal bodies for medical purposes, or for use in operative surgery upon the body or in preparation for operative surgery, together with devices designed to assist in operative surgery.

An endoscope may be an arthroscope (for joint surgery), a laparoscope (for abdominal surgery), colonoscope (rectum, colon, and lower small intestine), cystoscope (bladder and urethra), encephaloscope (brain), hysteroscope (vagina, cervix, uterus, and fallopian tubes), sinuscope (ear, nose, throat), thoracoscope (chest outside the lungs), tracheoscope (trachea and bronchi), esophageoscope (esophagus and stomach), etc.

### SUMMARY

In general, in a first aspect, the invention features an apparatus. The apparatus has a processor and a machine readable nontransitory memory. The memory has stored therein, instructions that, when executed, cause the processor to perform certain steps. Signals of voice utterances are accepted from a surgeon using an endoscope. Speech recognition technology decodes the voice utterance signals into commands to control the endoscope. Control commands are issued to components of the endoscope to implement the decoded voice utterances. Concurrently with the accepting and decoding of voice utterances, the processor accepts and decodes control signals from another input device. The processor issues control commands to components of the endoscope to implement the decoded other input control signals. The processor accepts the voice utterances and other input control signals concurrently and issues the control commands to the components concurrently.

In general, in a second aspect, the invention features an apparatus. The apparatus has a processor and a machine readable nontransitory memory. The memory has stored therein, instructions that, when executed, cause the processor to perform certain steps. Data are obtained from a database that stores properties specific to speech of a specific surgeon scheduled to used an identified endoscope, and/or information indicating spoken utterances associated with a specific procedure for which the identified endoscope is to be used. Signals of voice utterances are accepted from a surgeon using the identified endoscope. Speech recognition technology decodes the voice utterance signals into commands to control the endoscope. The decoding is based at least in part on the properties specific to speech of the specific surgeon. In addition or in the alternative, the decoding is based on information indicating spoken utterances associated with the specific procedure. Control commands are issued to components of the endoscope to implement the decoded voice utterances.

In general, in a third aspect, the invention features an apparatus. The apparatus has a processor and a machine readable nontransitory memory. The memory has stored therein, instructions that, when executed, cause the processor to perform certain steps. Data are obtained from a database designed to stores properties specific to speech of a specific surgeon scheduled to used an identified endoscope, and/or information indicating spoken utterances associated with a specific procedure for which the identified endoscope is to be used. Voice utterances signals are received from a surgeon using an endoscope. Speech recognition technology decodes the voice utterance signals into commands to control the endoscope. The decoding is based at least in part on the properties specific to speech of the specific surgeon, and/or information indicating spoken utterances associated with the specific procedure. Control commands are issued to components of the endoscope to implement the decoded voice utterances. Concurrently with the accepting and decoding of voice utterances, the processor accepts and decodes control signals from another input device. The processor issues control commands to components of the endoscope to implement the decoded other input control signals. The voice utterances and other input control signals are accepted concurrently, and the control commands to the components are issued concurrently.

Specific embodiments may feature one or more of the following. Voice utterance signals and control signals from two other input devices may be accepted and decoded concurrently. Control commands to implement the decoded signals, from the speech recognition technology and two other input devices, may be accepted and issued concurrently. The processor may accept and decode control signals from at least two of a pushbutton on the endoscope, a front panel of a control unit of the endoscope, and a foot pedal. Voice utterance signals and control signals from the other input device may yield overlapping sets of commands to components of the endoscope. The set of commands available by voice utterance may be disjoint from the set of commands available via the other input device. Voice utterance signals may be decoded into at least four categories from start and stop recording of video; raise and lower illumination; control water or gas insufflation; adjust image imaging parameters including at least two of contrast, saturation, color temperature, and white balance; increase or decrease zoom; change focal distance; horizontal and vertical pan; and rotate an image displayed on a monitor. Voice utterance signals may be decoded into at least five of those categories. Voice utterance signals may be decoded into at least three of those categories. The voice utterances may control water or gas insufflation via a voice utterance directed to distension or contraction of a surgical cavity. The issued control commands may be directed to increasing or decreasing of flow of insufflation fluid. The speech recognition technology may be trained to a single person's voice. The speech recognition technology may filter out sounds other than that single person's voice. An endoscope may have a microphone mounted therein. An endoscope may have a microphone and a pushbutton mounted adjoining each other. A database may provide data to be used by the speech recognition technology to improve decoding of the voice utterance signals. Data from the database may be obtained based at least in part based on an indication of identification of a specific endoscope. The identification of the identified endoscope may be obtained via a machine read of a machine-readable identification code of the identified endoscope. The database may further store operational properties of individual endoscopes. The processor may receive image data from an image sensor of the identified endoscope.

The above advantages and features are of representative embodiments only, and are presented only to assist in understanding the invention. It should be understood that they are not to be considered limitations on the invention as defined by the claims. Additional features and advantages of embodiments of the invention will become apparent in the following description, from the drawings, and from the claims.

### DESCRIPTION OF THE DRAWINGS

FIG. 1 is a block diagram of an endoscope system with a computer.

FIGS. 2A, 3B, and 3C are perspective or perspective cutaway views of endoscopes and/or endoscope related apparatus.

FIG. 2B is a flow chart.

FIG. 3A is a plan section view of components of an endoscope.

### DESCRIPTION

The Description is organized as follows.

| | |
|---|---|
| I. | Overview |
| II. | Voice control of an endoscope |
| | II.A. Voice control |
| | II.B. Implementation |
| III. | Machine-readable serial number as an assist to voice recognition |
| IV. | Voice control of liquid flow |
| V. | Embodiments |

### I. Overview

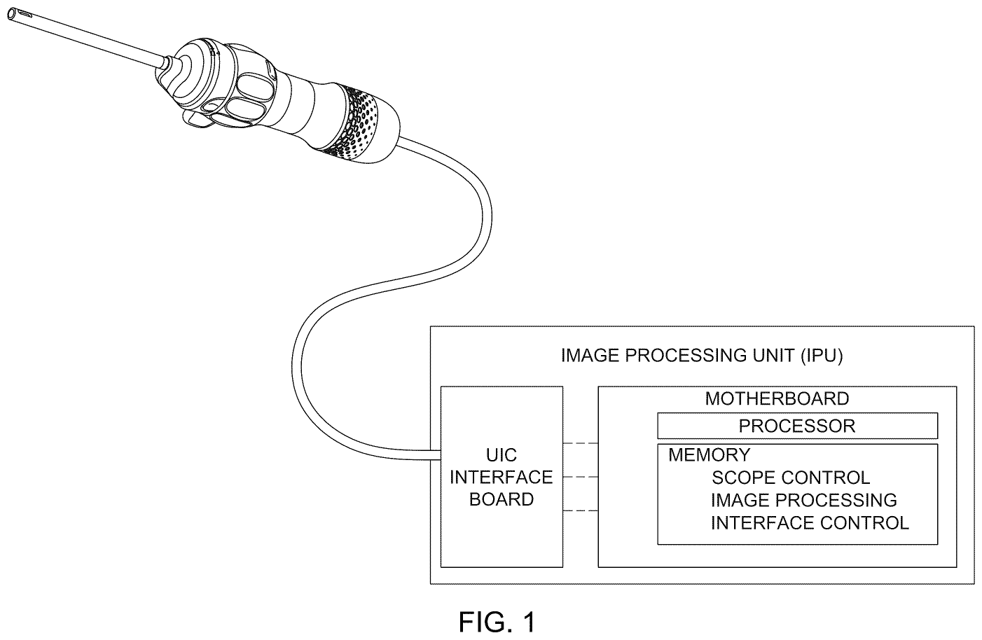

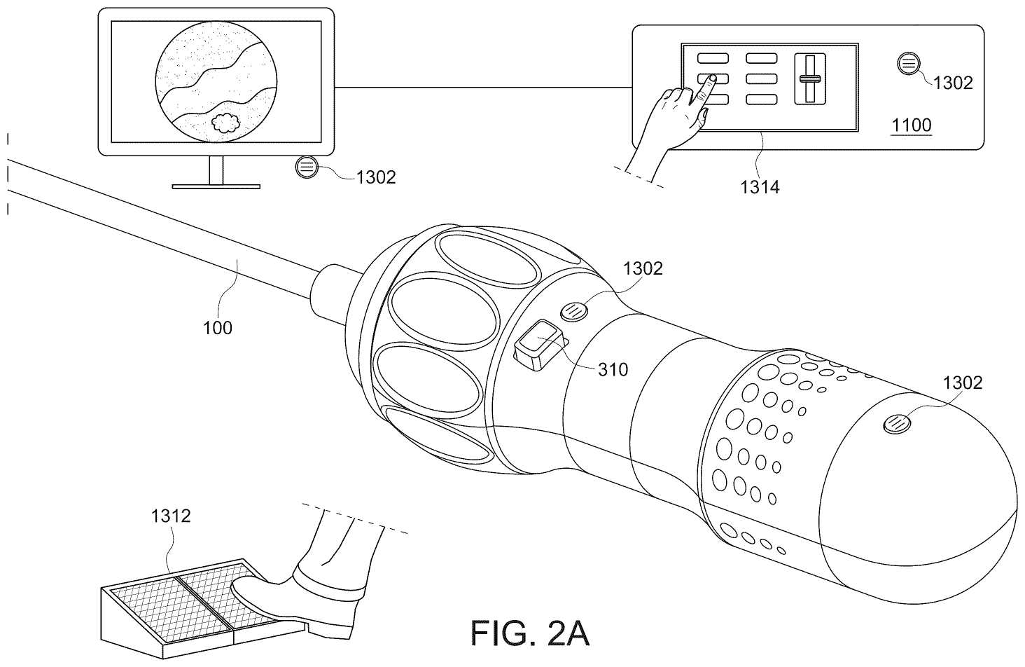

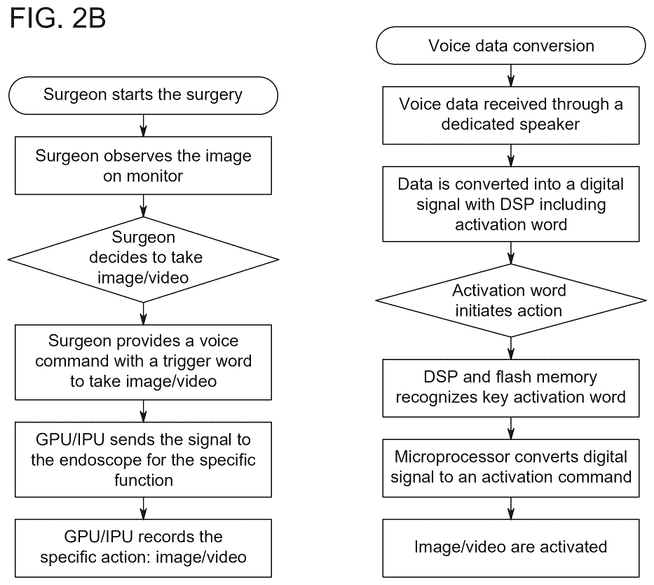

Referring to FIGS. 1, 2A, and 2B, endoscope 100 (such as an arthroscope, laparoscope, or other) may be used for joint surgery, joint access, or other minimally-invasive surgery. An endoscope may have voice control. The endoscope may be supported by a data processor that accepts video signals and displays the video on a monitor, and performs other control functions. During preparation for surgery, the processor may obtains voice training data for a specific surgeon, and/or information indicating spoken utterances associated with a surgical procedure for which the endoscope is to be used, for example, indicting the jargon for that surgery, so that the voice recognition system knows that related voice utterances are more likely than other utterances. During surgery, the processor may process voice utterances signals from a surgeon and decode the voice utterances signals into commands to control the endoscope. The decoding may be based at least in part on the data obtained before surgery. Concurrently with the accepting and decoding of voice utterances, the processor accepts and decodes control signals from at least one other input device. The processor issues control commands to components of the endoscope to implement the decoded other input control signals. The voice utterances are accepted, decoded, and executed concurrently with the other input control signals.

Referring to FIG. 1, endoscope 100 may be part of an overall system designed to deliver high-definition video for use in endoscopic surgeries. A scope controller or image processing unit (IPU) 1100 may be configured to acquire images or signals from a camera or image sensor of the endoscope, typically mounted at the tip. The digital scope controller converts the signal or image from the image sensor into a processed digital image and transmits the processed signal to a monitor on which it is displayed. The system may provide live high-definition video to be displayed on a video monitor, and to be captured as stored video and still images; illumination of the surgical cavity, irrigation and/or inflation (insufflation) of the surgical site, and image refinement such as zoom, panning, rotation, adjustment of illumination, brightness, and/or contrast, removal or reduction of hotspots and other artifacts, etc. In a configuration where the scope is sold as a disposable single-use configuration, with an electronic serial number that ties back to calibration factors measured at the factory (see § III and ¶¶ [0030] to [0035], below), the scope may be individually calibrated, and that calibration data may be retrieved by a lookup based on the electronic serial number.

### II. Voice Control of an Endoscope

### II.A. Voice Control

Referring to FIG. 2A, voice control may allow a user to interact with smart devices using spoken commands, by adding voice control to an endoscope system. Microphone 1302 may be added, typically mounted with its peak sensitivity axis pointed toward the surgeon's mouth. Microphone 1302 may be mounted near a pushbutton 310 because button 310 tends to stay pointed in the direction of the surgeon's face. A voice recognition system may be hosted in any available microprocessor, typically the microprocessor in the IPU (image processing unit) box 1100.

Voice processing may permit a surgeon to command the endoscope by speaking an utterance to a micriophone, which sends sound signals to the endoscope's signal processor. The endoscope's audio signal processor may transmit audio signal of the utterance to IPU 1100. IPU 1100 may process the utterance's audio signal to determine whether any functionality of the endoscope is intended by the surgeon's utterance. If the voice utterance is recognized as a command, IPU 1100 may issue one or more appropriate control signals to the appropriate component of the endoscope to implement the spoken command. For example, IPU 1100 may recognize utterances and control scope components to activate image or video capture, stop image capture, raise or lower illumination, start or stop water or gas insufflation, adjust image imaging parameters like contrast, saturation, brightness, color temperature, depth of field, exposure, increase or decrease zoom, pan image on horizontal or vertical axes, rotate the image, perform white balancing, etc. In some cases, the speech recognition system and the alternative input method may control the same or overlapping parameters: for example, the function of capturing a still frame may be available via both pushbutton 310 and via voice recognition, or raising brightness of illumination may be available via all three of foot pedal 1312, front panel 1314 of IPU 1100, and voice recognition. In other cases, different commands may be segregated by input mode.

Voice commands may have one or more of the following advantages over pushbutton 310 and other similar physical command modalities. By separating different kinds of actions onto separate sensory or motor channels and modalities, interference and risk of error may be reduced-just as humans are more effective when different kinds of information are separated into visual and auditory modalities, the risk of error may be reduced if hand motions are confined to guiding the endoscope, and the commands for image control are separated to a voice channel. In addition, an endoscope designed to receive voice commands can receive commands from multiple personnel in an operating room, including the surgeon, the anesthesiologist, nurses, and/or other technicians, as opposed to a single pushbutton 310 that can only be accessed by a single person. For example, the surgeon may give one voice command “increase brightness,” and a press of button 310 or footpedal 1312 to capture a still frame, while a support technician/nurse is giving a command at front panel 1314 of IPU 1100 to store data in the patient's medical record, or the anesthesiologist requests a change of display of the patient's vital signs, or change in the anesthesia drip. The voice control system may be implemented via field-upgradeable software or firmware, with configuration parameters that may be tailored or optimized to an individual surgeon's preferences. In cases where pushbutton 310 is on the endoscope itself, during use pushbutton 310 may become not easily accessed by the surgeon's finger, for example, because of rotation. Because voice commands are less sensitive to position and orientation, voice commands may be accessible and form a reliable backup input mode when button 310 rotates out of easily accessible position.

Additionally or alternatively, the overall system may enable multiple command modes concurrently. For example, in some embodiments, the endoscope control system may be designed to accept commands through both voice control and physical interfaces 310, 1312, 1314 concurrently. This dual-control approach may enhance the surgeon's flexibility and may ensure reliable operation in various scenarios. Voice control provides hands-free convenience and can handle complex commands, to complement the simplicity and reliability of physical interfaces 310, 312, 1314, especially in environments where voice recognition may be challenging.

Endoscope device 100 may be equipped with a unified endoscope control system 1100 that can process inputs from both voice commands and from physical interfaces, such as a button on the scope, a foot pedal 1312 for the surgeon, and commands at the front panel 1314 of IPU 1100 box. This system ensures that commands from any source are recognized and executed seamlessly. In some cases, each of the input devices 310, 1302, 1312, 1314 may generate an event packet in a queue, and then controller IPU 1100 may take those event packets from the queue sequentially and execute them. This may ensure that commands issued nearly-simultaneously by two different input devices 310, 1302, 1312, 1314 do not generate race conditions and are handled without conflict.

In some versions, the voice control system and pushbutton 310 are configured to control different aspects of the endoscope operation. For example, pushbutton 310 can be used to control camera angle, zoom, focus area, and other attributes, while voice commands 1302 can be used to instruct the camera to take pictures, and control insufflation. In other versions, voice control may enable the device to respond to any command issued by voice or by pushbutton action and can be programmed to prioritize inputs based on specific criteria. For example, in a noisy environment where voice commands might be misinterpreted, the endoscope control system may be programmed to recognize only specific voices to filter out the ambient noise, and/or may be programmed to prioritize physical button inputs.

In some cases, a voice command may translate directly to a control signal. For example, “brighter” or “zoom in” or “take picture” may result in a single direct control signal. In other cases, a single voice command may be translated into a derivative control signal, or a series of control signals. For example, a voice utterance “increase inflation” may be translated into control signals to increase the fluid flow from an injection-side pump, and then three seconds later return to the same injection pumping rate (while holding the evacuation-side flow constant) (see § IV at ¶¶ [0040] to [0045]). Alternatively, “increase inflation” may be translated into a command to slow or restrict flow on the evacuation-side pump for five seconds (while holding the injection flow rate constant). Either of these translations of a single voice utterance into multiple commands implements the requested utterance, to increase inflation or distension of the surgical cavity.

### II.B. Implementation

Microphone 1302 may be added to the endoscope system. In some cases, microphone 1302 may be added to endoscope handle 100. In some cases, microphone 1302 may be added on, at, or near the proximal end of the endoscope handle, facing toward the surgeon's mouth. In some cases, microphone 1302 may be added to the proximal end of the endoscope handle, as opposed to the outer surface near the proximal end, to ensure that it will not be covered by the surgeon's hand. In some cases, microphone 1302 may be added on, at, or near the proximal end of the endoscope handle facing toward the likely location of the surgeon's mouth. In some cases, microphone 1032 may be mounted on handle 112, 114 near button 310. In some cases, microphone 1302 may be mounted on a boom or lamp over the surgical site. In some cases, microphone 1302 may be mounted on a wall or other fixed location, or some other location outside the sterile field. In some cases, microphone 1302 may be a headset, or a microphone mounted in the surgeon's mask. Microphone 1302 may be connected into the rest of the system via wireless connection, or via a copper wire.

Integrating microphone 1302 into endoscope 100, particularly near, at, or on the proximal end, may offer one or more of the following advantages. Placing microphone 1302 in endoscope itself 100 may reduce or even eliminate the need for further equipment, thereby reducing risk of infection, reducing the need to train operating room staff to set up an additional piece of equipment, reducing the need for separate sterilization, reducing installation inconvenience and error, and reducing training overhead. Placing microphone 1302 in endoscope itself 100 may take advantage of the established wired communication already present between endoscope 100 and IPU 1100, as opposed to adding another wire from a headset or similar other microphone location. Placing microphone 1302 in the endoscope itself 100 may take advantage of an existing tether or umbilical cable for insufflation liquid or gas, or electrical cable for power and signal, may reduce change to requirements for sterilization, setup, and workflow. The proximal end of endoscope 100 tends to be the apparatus that is closest to the surgeon's mouth during a procedure, thereby reducing interference from background noise in the operating room. Positioning microphone 1302 on the endoscope handle may be especially advantageous in a scope 100 designed for single-use disposability. Microphone 1302 may be a moving coil, ribbon, condenser, or other microphone technology. Microphone 1302 may be disposable, to reduce sterilization issues.

The microphone's audio signal may be transmitted as an analog audio signal to an analog-to-digital converter in the endoscope handle or in IPU 1100. Microphone 1302's audio signal may be digitized by a processor either in the endoscope handle or by IPU 1100, and sent as a digital stream to IPU 1100. If the endoscope has an existing tether or umbilical cable for insufflation liquid or gas, or electrical cable for power and signal for other components of the endoscope, a dedicated conductor may be added for the audio signal, or (especially if the signal is digitized) audio signal packets may be multiplexed onto an existing conductor with other signal packets. Avoiding new cabling, or piggybacking new signal on old cable, may avoid change to requirements for workflow, sterilization, setup etc. IPU 1100 may manage the overall operation of the voice control system and endoscope system. IPU 1100 may coordinate communication between microphone 1302, processor, and other components of the smart endoscope. IPU 1100 may also handle connectivity to external networks or cloud services, enabling more advanced processing and access to cloud-based databases for improved voice recognition accuracy.

The voice control system may leverage natural language processing (NLP) and voice recognition technologies to interpret and execute user instructions. To enable such functionality, microphone 1302 captures sound waves, including the user's voice, and converts them into analog electrical signals. These analog signals are then converted into digital signals using an analog-to-digital conversion. The processor then operates to filter out noise and enhance the quality of the voice signal. This step may involve techniques like noise reduction, echo cancellation, and gain control. The processor extracts relevant features from the voice signal, such as pitch, tone, and phonemes. These features may be used to recognize and distinguish different words and phrases The extracted features may be compared against a database of known patterns using algorithms like Hidden Markov Models (HMM) or neural networks to identify spoken words. Once the words are identified, NLP algorithms interpret the meaning and intent behind the spoken command. This involves understanding context, syntax, and semantics. Based on the interpreted command, the smart device performs the requested action, The device may provide auditory or visual feedback to confirm the action has been completed.

Voice recognition may be performed by a commercial voice recognition product such as Apple's Sire, Amazon's Alexa, Microsoft's Cortana, Google's Assistant, Dragon Naturally Speaking, or others. Voice recognition software may be hosted in a microprocessor in the endoscope, in a microprocessor in the endoscope's IPU, or in a remote computer accessible over the cloud.

Referring to FIG. 2B, when a surgeon begins a procedure, video signal from the endoscope may be displayed on a monitor (steps 1321, 1322). The voice recognition system, via microphone 1302, may continuously listen for a wake word and once the wake word is detected, the device activates the voice recognition system. Alternatively, the voice recognition system may be activated in advance. The repertoire of recognized utterances may track the controllable functions of the components of the endoscope. For example, commands to an endoscope named “Summit” may be “Summit take picture” or “Summit start video” or “Summit start video record” or “Summit stop video” or “Summit illumination brighter” or “Summit lavage” or “Summit pan camera up” or “Summit inflate.” The voice recognition software may be trained to recognize multiple synonyms for the semantically-identical command. The spoken command is then translated into one or more control signals, which may then be then placed it a software queue of control signals from all input devices or generated internally, which may then be dequeued for execution. For example, the flash rate and duration of the illumination LED may be controlled by the control board in the endoscope handle. Command execution may involve sending command signals from IPU 1100 to the handle control board. Image cropping and rotation may be handled by the video pipeline on IPU 1100, so command execution may involve sending updated video processing parameters to that video pipeline.

The voice recognition software may be trained to a specific surgeon, so that it can prioritize utterances from the surgeon over utterances of others. The software may permit an individual surgeon to set individually-preferred utterances for commands. The voice recognition software may be tuned or trained to reduce sensitivity to ambient noise in the operating room.

### III. Machine-Readable Serial Number as an Assist to Voice Recognition

Each scope as shipped may have one or more scope-specific data encoded in machine-readable and scannable, and/or human-readable form. The data may include one or more of the scope's serial number, configuration data, manufacturing calibration data, tracking data, etc. These data may be used for multiple purposes. Greater detail may be found in US20240355465A1, Information Management and Inventory Tracking for Medical Devices, incorporated by reference. Each machine-readable serial number may be related to information describing specific configuration and/or capability information relating to the scope. This information can be checked as the scope is paired to a patient or a similar time when specific capabilities of a specific scope or component should be compared to the specific needs of a patient. The serial number may be used to associate with a patient and to track inventory. The same database may relate individual scope ID's to either surgeon voice training data or to data that assists in voice recognition.

During procedure setup, an electronic serial number of the specific scope to be used may be scanned. This serial number may tie to database records with information about the specific scope, the specific procedure scheduled for that scope, the specific surgeon scheduled, and the specific procedure. This combination of information, gathered shortly before the procedure begins, may permit improvement of surgery and voice control in several ways, such as:
The database may have training data for a specific surgeon's and/or specific anesthesiologist's voice and speech. This training data may improve speed and accuracy of the system's speech recognition.
The database may record the specific ways this surgeon does this specific procedure, such as which instruments this surgeon prefers, specific anesthetic agents preferred by the anesthesiologist, and the like. Knowing in advance which words are more likely may improve speed and accuracy of speech recognition.

Information may be encoded in a machine-readable code on packaging for the scope, embedded in packaging, or embedded in the scope as a scannable code. The scannable code may be any form of Matrix (2D) or linear bar or machine vision code that can be scanned by a smartphone. Examples include any variant of QR code, Code 39, Code 49, Code 93, Code 128, Aztec code, Han Xin Barcode, Data Matrix code, JAB Code, MaxiCode, PDF417 code, SPARQCode, and others. The scannable code may be an RFID or similar tag that can be scanned by a sensor in a phone. The scan may be optical, or may use any IEEE 802 or related communications protocol, including Bluetooth, RFID (ISO 14443) or NFC (ISO 18092). The scannable code may be coded on packaging, in the scope's handle, or in the nose cap of a replaceable scope insertion tip. Alternatively, it may be stored in an EEPROM memory in the handset, connected by an SPI (Serial Peripheral Interface), I2C (Inter-Integrated Circuit), USB, or a one-wire protocol, to be read when the scope is plugged into the image processing unit (IPU). The scope may have a small amount of non-volatile memory that can be read and written during initial device manufacture and by IPU 1100. That memory may store an electronically-readable serial number written into the memory during manufacture. This memory may also store per-scope configuration information, such as scope model, serial number, white balance coefficients, lens properties that can be corrected in IPU 1100, focus parameters, etc. This memory may also be used to store usage information such as timestamps or usage time as determined by IPU 1100, to prevent reuse of the scope after 24 hours. To ensure tamper resistance, information written into the handle memory may be written under a secure or encrypted protocol used between IPU 1100 and the handle's microprocessor.

The information may be stored as s single datum (essentially a serial number, or some other datum that semantically-equivalently uniquely identifies the scope), which may be used as an index key it a database at a server, which in turn has the full data about the scope. In some cases, various operating parameters of the scope may be stored in a database of a server, and either the model number or serial number may be used as a lookup key to retrieve this configuration data and collection of parameters. In other cases, the operating parameters may be separately individualized to each individual scope. For example, at the beginning of an arthroscopic surgery on a shoulder, IPU 1100 may confirm that the scope to be used is indeed an arthroscope of suitable diameter, length, and optical capabilities. The two approaches may be combined, so that some parameters are stored based on model number, and others are stored individually per scope.

Data stored in on-board memory or in a remotely-accessible database may include:
A unique serial number or database lookup key
A model number and version number (integer or ASCII)
A text description of the component' Model Number/Model identifier that can be displayed on the control display screen (typically a 32-charactarer ASCII string)
Calibration/normalization data
Full configuration specifications—for example:
   The manufacturer's part number for the image sensor, which may allow many additional properties of the image sensor to be looked up in a table in IPU 1100 including:
      The size (in rows×columns) of the image sensor
      Supported frame rates for the sensor and frame reporting rates
      Minimum/maximum integration time and integration time configuration resolution (for example. 0.1 to 100 ms in increments of 1 ms)
   An identifier for illumination sources on board the scope—white, infrared, ultraviolet, individual colors, image plane resolution (720×480, 1280×720, etc.)
   An identifier for what sensors are in the image plane—for example, one bit on/off for each of red, green, blue, ICG infrared, and other colors as extended in future software updates
   Information to establish white balance, color correction gamma curves, coefficients for distortion correction, sensor color sensitivity, illumination color, etc.
   (Boolean) Does/does not provide an illumination source in the handpiece
   (Boolean) Does/does not provide a de-fogging heater in the handpiece
   (Boolean) Does/does not provide a rotation sensor in the handpiece
   (Boolean) Does/does not support focus control in the handpiece
Calibration/normalization data—for example
   Corrective data for variations in lens focus
   Correction coefficients to compensate for image sensor color sensitivity, illumination color, white balance, distortion correction
   LED illumination brightness coefficients
An identifier to enable/disable certain image enhancement parameters based on the hardware image configuration—this may be used to pre-configure image processing settings based on anticipated imaging application for this scope. For example, a bit vector may enable or disable optimization of resolution, contrast, smoothing, and other optical properties
Various size and length properties, which may be important to control water pressure and the like
Manufacturing date
Date and time of first use
Date and time of initial connection and disconnections
Length of scope use during the procedure

The connector may be a standard connector (e.g. USB-A) or a special purpose connector. A special purpose connector may ensure that mismatched devices are not plugged together. A special-purpose connector may allow additional pins to support all required signals and video, for example, video signals over twisted pair, higher current to power to a heater in the handset, and an optical connector for illumination light fibers.

The database may have tables describing scopes, facilities, and the like. One table may describe properties of models of scope, properties that are common to a product ID class of scopes. A second table may describe individual scopes.

The database may have tables describing procedures. One table may describe properties of a class of procedures. A second table may describe individual procedures on individual patients.

Table procedure_type

| Field | Type | Data Included |
|---|---|---|
| procedure_name | text | name of procedure |
| billing_code | integer | AAPC/CPT/HCPS category code |
| [other] | | links to table “scope_product_id” for scopes useable |
| | | for this procedure, surgeons and facilities authorized to |
| | | perform this procedure, etc. |
| status | integer | status |
| speech_assist | relation | data describing words likely to be used during this |
| | | procedure, and thus more likely to be used and |
| | | recognized by the speech recognition system |
| is_deleted | boolean | |

Table procedure

| Field | Type | Data Included |
|---|---|---|
| procedure_type | relation | link to row of table “procedure_type” |
| procedure_name | text | Name of procedure (if different than the name from |
| | | “procedure_type”) |
| patient | relation | link to a row of table “patient” for the patient of this |
| | | procedure |
| scope | relation | link to a row of table “scope_device” for the specific |
| | | scope used in this procedure |
| facility_id | relation | link to a row of table “facility” for the facility at which |
| | | the procedure is performed |
| procedure_date | date | date on which the procedure performed |
| status | integer | status |
| surgeon_id | relation | link to a row of table “surgeon” |
| is_deleted | boolean | |

Table surgeon

| Field | Type | Data Included |
|---|---|---|
| surgeon_voice_training | relation | link to data for voice training of the speech recognition |
| | | technology that trains it to this specific surgeon |
| is_deleted | boolean | |

The database may have tables that set up the relations between scopes, procedures, patients, surgeons, inventory states, and the like.

An architecture of a system may be arranged around client-server principles. Users may interact with user-device client software, such as browser HTML or a downloaded app on either a phone or a desktop computer. On the server side, the front-end may be provided by off-the-shelf components such as Amazon AWS, ALB (application load balancer), and/or CloudFront content delivery network. The application server may include components for patient management, inventory management, management of user IDs, permissions, authentication, and authorization; facility management; procedure management; order management; scope management; pairing between scopes and patients and procedures, and other administrative functions. A relational/SQL database may provide storage and lookup.

### IV. Voice Control of Liquid Flow

Safety of an endoscopic procedure depends on clarity of view of the surgical field. Irrigation systems inject fluid into a surgical cavity (for arthroscopic surgery, the joint capsule) to replace synovial fluid and distend the joint to improve visibility of the surgical area. The pressure exerted by the fluid distends the surgical cavity (such as the joint space), while the fluid itself flushes out pre-existing debris from the pathology, blood, ablation or shaver debris from the surgery, and to keep the soft tissues apart. Fluid pressure above the patient's blood pressure tends to control bleeding. Saline (0.9%) or lactated Ringer's are common irrigation solutions.

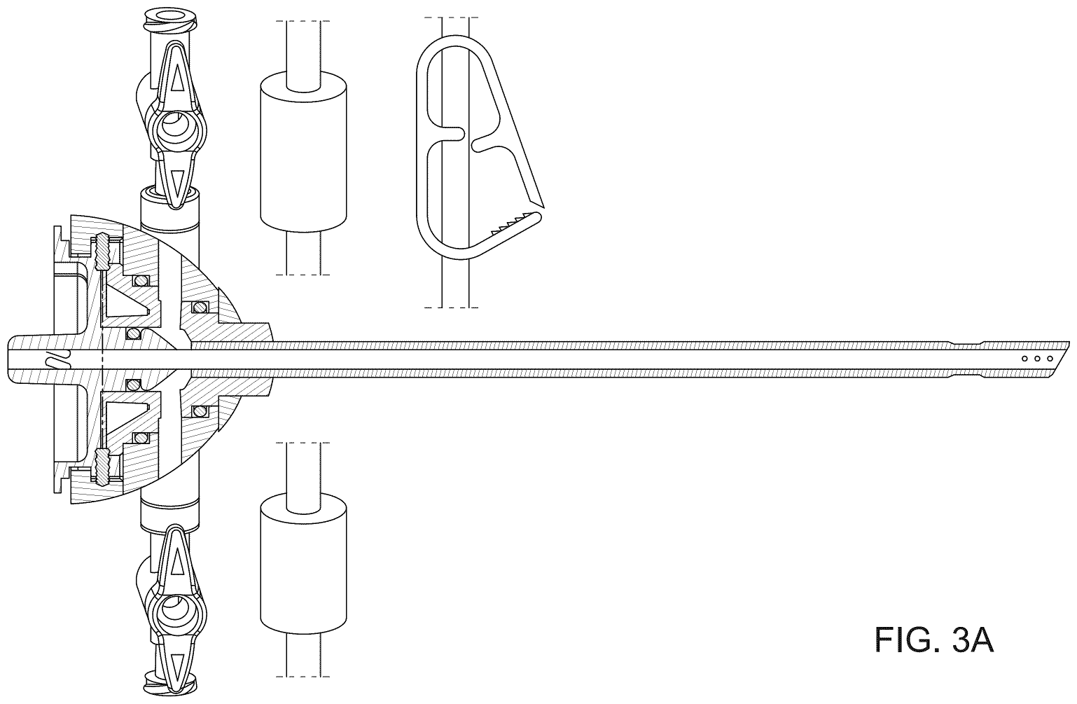

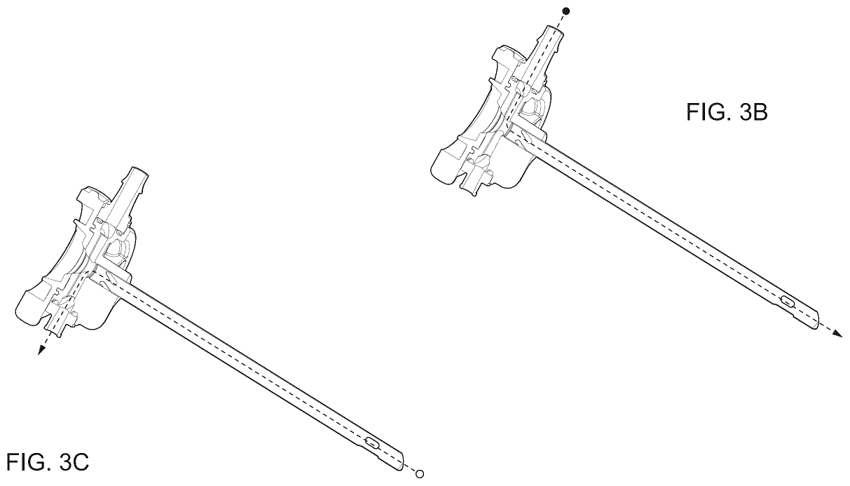

Referring to FIGS. 3A, 3B, and 3C, hoses for injection and evacuation of irrigation and inflation fluids may be attached to a distal end of the handle of the endoscope. Fluids flow into the surgical cavity from a hose though the insertion shaft and out the tip. This hose may have a hose clamp to control flow, and may attach to the scope at a barb connector. Fluids may be injected from the endoscope into the surgical cavity on an injection side, and out to waste though an evacuation side. The evacuation hose may be connected to the scope via a luer lock. Any leakproof connector may be used, and within those options, the connector may be chosen for ease of assembly. The chamber between the two hoses and the insertion shaft may form a manifold in the range of 10 ml or more. This manifold is desirably kept as small as reasonably possible, in order to reduce mixing of clean incoming irrigant and evacuated irrigant.

In some cases, the fluid-handling assembly of FIGS. 3A to 3C may be disposable, packaged with the rest of the endoscope assembly 100. Because the injection side of the fluid-handling system may become contaminated by patient tissues and fluids from the evacutation side, sterilization may be difficult. Packaging the fluid-handling assembly of FIGS. 3A to 3C with the rest of the endoscope system may reduce set-up time and inventory management.

The fluids flow into and out from the surgical cavity by traveling down passages in insertion shaft. In some cases, as shown in FIGS. 3B and 3C, the insertion shaft may have a single lumen shared for both injection and evacuation, so injection and evacuation must be sequenced. In some cases, the insertion shaft may have multiple fluid flow passages so that injection and evacuation can be concurrent. The passages may be molded into the surgical shaft, for example, by extrusion (see U.S. Pat. Nos. 9,364,204 and 10,694,927, incorporated by reference). In some cases, the fluids may flow around wiring or a circuit board that mounts camera 410 and/or illumination LED. In some cases, the circuit board may be the divider that separates the injection passage from the evacuation passage. Electrical power to camera 410 may be supplied over conductors in a flexible cable or on a printed circuit board (flexible or rigid), and may be insulated with a conformal and insulating coating such as parylene. The design seeks to ensure that the most-constricted point of the route from the injection hose to the tip, and back from the tip to the evacuation hose, is of large cross section to provide adequate fluid flow volume. In some cases, insertion shaft may have lumens or working channels for the introduction of surgical instruments, such as laparoscopic instruments or colonoscope snares or snips.

Referring to FIG. 3A, in other variants, both sides of the cannula may use luer locks. One or both sides may use barb connections. One or both sides may use stopcocks. One or both sides may use clamps. In some cases, the pressure on the injection side may be supplied passively, driven by gravity feed of a by hanging an IV bag at a desired height above the joint. In some cases, one or both sides may include one or more motorized pumps, such as a peristaltic pump, lobe gear pump, roots style pump, centrifugal pump, axial flow pump, diaphragm pump, or impeller pump. The injection and evacuation regulators may be different. For example, one side may have a motor-driven pump and the other side may be passive flow. On the injection (into the surgical site) side, a stopcock may constrict fluid flow, so a hose clamp may be the preferred closure. Pumps or other flow regulation apparatus may be built into a common housing with a connector between endoscope shaft and handle. The evacuation side may have filters for trapping evacuated tissue debris or smoke.

In some cases, other surgical tools (such as the shaver or ablation tool) may have their own suction systems, controlled by IPU 1100 and voice control. In these cases, the fluid supplied from inlet hose may flow into the surgical cavity continuously, and be suctioned out at the other device. In some cases, the assembly may include both an injection pump on the injection side and a evacuation pump on the evacuation side, connected in series to each other. By injecting clear fluid at the endoscope, and removing bloody or contaminated fluid at the other device, clarity of view may be maintained. The hose clamp or stopcocks or pump motors may be used to moderate fluid flow volume. The endoscope may have a pressure sensor at or near the optical tip, or in the injection channel, or in the evacuation channel. These pressure sensors may provide signal to a controller that computes control signals to be sent to pump motors or valves to control flow into the surgical site and out to exhaust. In some cases, flow regulation pump motors or valves on the injection and/or evacuation side may control flow rates on the fluid flow into the joint and flow out. Flow may be controlled as fluid volume rate or as pressure. Flow rates (volumetric or pressure) may be controlled, and flow in and evacution flow out may be separately controlled to maintain a desired differential, to establish or maintain one or more of optimum pressure, cavity volume, and/or joint distension. The control may ensure against over-pressurizing to the point of causing extravasation injury. The evacuation side may be controlled to provide suction, or to provide backpressure to control inflation of the surgical cavity. The evacuation side may include a pressure limiting valve or pressure relief valve or additional sensors or pressure gauges to regulate force or pressure to be applied to ensure patient safety. The flow regulator may be controlled by computer control, based on the pressure sensor reading and a feedback-driven control algorithm to maintain fluid volume or pressure within a range specified for the surgery. Flow may be under voice control, as discussed in § II at ¶¶ [0016]-[0029]. For example, the voice control system may accept commands such as “increase distention” “reduce irrigation flow,” “clear smoke,” and the like, and the controller may adjust the injection and evacuation flow rates to implement the command. Placing the pumps close to the surgical site (as opposed to several meters away as part of the controller and the rest of the support tower) may improve the precision of flow control, and reduce kinking of hoses. In some cases, the pump motors may be located within the handle. Control of the pump motors may be by signal wire, optical fiber, or wireless. The irrigation fluid may be liquid (saline, Ringer's solution, etc.) or gas (nitrogen, carbon dioxide, etc. with or without a desired level of oxygen) or may combine or alternate between. The system may also include procedure templates stored as blocks in a look up table for presets for different procedures such as knee, shoulder or hip arthroscopy as well as the ability to store custom user preference settings. During setup for a procedure, the electronic serial number (see § III at ¶¶[0030] to [0035]) may be used to select a template block of parameter presets.

### V. Embodiments

Embodiments of the invention may include any one or more of the following features, singly or in any combination.

An apparatus may include a computer processor and a memory. The processor is programmed to receive video image data from an image sensor at the distal end of an endoscope and to display the image data to a surgeon in real time. The processor is programmed to process the image data received from the image sensor via a machine learning model, the machine learning model trained to simultaneously upsample the image data to a resolution higher than that captured by the image sensor, to sharpen edges, and to enhance local contrast.

An apparatus may include a computer processor and a memory. The processor is programmed to receive video image data from an image sensor at the distal end of an endoscope and to display the image data to a surgeon in real time. The video image data have a frame rate at which the image data are generated by the image sensor. The processor is programmed to control the image sensor and/or an illumination source designed to illuminate a scene viewed by the image sensor, the controlling programmed to underexpose or overexpose every other frame of the video image data. The processor is programmed to process the image data received from the image sensor to combine successive pairs of frames of the image data to adjust dynamic range to enhance over-bright or over-dark portions of the image to expose detail, and to generate combined frames at the full frame rate of the video as generated by the image sensor.

An apparatus may include a computer processor and a memory. An apparatus may include a computer processor and a memory. The processor is programmed to receive video image data from an image sensor at the distal end of an endoscope and to display the image data to a surgeon in real time. The processor is programmed to sum an error for an intensity of the image relative to a setpoint intensity. The processor is programmed to simultaneously control at least two of gain, exposure, and illumination via a PID control algorithm to achieve image display at the setpoint intensity, maximum change per step of the PID control damped to prevent oscillation.

Embodiments may include one or more of the following features, singly or in any combination. The processor may be further programmed to control the image sensor and/or an illumination source designed to illuminate a scene viewed by the image sensor. The controlling may be programmed to underexpose or overexpose every other frame of the video image data. The processor may be further programmed to process the image data received from the image sensor to combine successive pairs of frames of the image data to adjust dynamic range to enhance over-bright or over-dark portions of the image to expose detail. The processor may be further programmed to generate combined frames at the full frame rate of the video as generated by the image sensor. The processor may be further programmed to sum an error for an intensity of the image relative to a setpoint intensity. The processor may be further programmed to simultaneously control at least two of gain, exposure, and illumination via a PID control algorithm to achieve image display at the setpoint intensity. A maximum change per step of the PID control may be damped to prevent oscillation. The processor may be further programmed to process the image data received from the image sensor via a machine learning model, the machine learning model trained to simultaneously upsample the image data to a resolution higher than that captured by the image sensor, to sharpen edges, and to enhance local contrast. The processor may be further programmed to enhance the video image data via dynamic range compensation. The processor may be further programmed to adjust exposure time, illumination intensity, and/or gain in image capture to adjust exposure saturation. The processor may be further programmed to enhance the video image data via noise reduction. The processor may be further programmed to enhance the video image data via lens correction. The processor may be further programmed to in addition to resolution, enhance at least two of dynamic range compensation, noise reduction, and lens correction. The processor may be further programmed to rotate the image display to compensate for rotation of the endoscope. The processor may be further programmed to adjust exposure time, illumination intensity, and/or gain in image capture to adjust exposure saturation.

An endoscope has an image sensor designed to gather photons to produce video. The image sensor is of a type that has properties that vary from one sensor to another within manufacturing tolerances. The endoscope has a connector to connect the endoscope to a computer image processor. The image processor is programmed to obtain data from a database designed to store properties of the endoscope's image sensor and/or the image sensor's behavior, the database storing properties specific to specific sensors or a specific class of sensors. The obtained data describes the image sensor's properties from the database. The image processor is programmed to compute normalized video based on the obtained properties and the video from the image sensor.

A computer processor is programmed to receive image date from an image sensor of an endoscope, and to obtain data from a database designed to store properties of endoscopes' image sensors and/or the image sensors' behavior. The image sensor is of a type that has properties that vary from one sensor to another, the database storing properties specific to specific sensors or a specific class of sensors. A computer computes normalized video based on the obtained properties and the video from the image sensor.

When a medical device or endoscope may be connected to its control computer, the computer may be programmed to obtain properties of the connected device from a database. The identification code may be a machine-readable 2D optical code. The identification code may be stored in machine-readable electronic memory. The database stores information relating to a device model of a class of medical devices manufactured to be interchangeable. The database may be designed to index to the device model information based on the read identification code of an individual medical device. The database may be designed to store data describing a color spectrum capability of the image sensor. The database may be designed to store data describing an image plane resolution of the image sensor. The database may be designed to store calibration properties of the endoscope's image sensor specific to the specific image sensor of the specific endoscope. The image processor may be programmed to compute normalized video based on the obtained calibration properties and the video from the image sensor. The calibration properties may describe a white balance of the image sensor, or a color correction gamma curve of the image sensor, or a distortion correction of the image sensor, or any two or more, or any three or more of these properties. The computer may compute a prediction of delivery date based on the read 2D identification codes. The computer may update the physical location and/or ownership database records to show current physical location based on the read 2D identification codes or read of the machine-readable memory.

A processor may accept signals of voice utterances from a surgeon using an endoscope. Speech recognition technology may decode the voice utterance signals into commands to control the endoscope. Control commands may be issued to components of the endoscope to implement the decoded voice utterances. Concurrently with the accepting and decoding of voice utterances, the processor may accept and decode control signals from another input device. The processor may issue control commands to components of the endoscope to implement the decoded signals from the other input device(s). The processor may accept the voice utterances and other input control signals concurrently and issue the control commands to the components concurrently.

Data may be obtained from a database that stores properties specific to speech, or speech training data, of a specific surgeon scheduled to used an identified endoscope. Alternatively or in addition, the database may store information indicating spoken utterances associated with a surgical procedure for which the identified endoscope is to be used. Signals of voice utterances may be accepted from a surgeon using the identified endoscope. Speech recognition technology may decode the voice utterance signals into commands to control the endoscope. The decoding may be based at least in part on the properties specific to speech of the specific surgeon. In addition or in the alternative, the decoding may be based on information indicating spoken utterances associated with the specific procedure. Control commands are issued to components of the endoscope to implement the decoded voice utterances.

Data may be obtained from a database that stores properties specific to speech, or speech training data, of a specific surgeon scheduled to used an identified endoscope. Alternatively or in addition, the database may store information indicating spoken utterances associated with a surgical procedure for which the identified endoscope is to be used. Signals of voice utterances may be received from a surgeon using an endoscope. Speech recognition technology may decode the voice utterance signals into commands to control the endoscope. The decoding may be based at least in part on the properties specific to speech of the specific surgeon. In addition or in the alternative, the decoding may be based on information indicating spoken utterances associated with the specific procedure. Control commands are issued to components of the endoscope to implement the decoded voice utterances. Concurrently with the accepting and decoding of voice utterances, the processor accepts and decodes control signals from another input device. The processor issues control commands to components of the endoscope to implement the decoded other input control signals. The voice utterances and other input control signals are accepted concurrently, and the control commands to the components are issued concurrently.

Voice utterance signals and control signals from two other input devices may be accepted and decoded concurrently. Control commands to implement the decoded signals, from the speech recognition technology and two other input devices, may be accepted and issued concurrently. The processor may accept and decode control signals from at least two of a pushbutton on the endoscope, a front panel of a control unit of the endoscope, and a foot pedal. Voice utterance signals and control signals from the other input device may yield overlapping sets of commands to components of the endoscope. The set of commands available by voice utterance may be disjoint from the set of commands available via the other input device. Voice utterance signals may be decoded into at least four categories from start and stop recording of video; raise and lower illumination; control water or gas insufflation; adjust image imaging parameters including at least two of contrast, saturation, color temperature, and white balance; increase or decrease zoom; change focal distance; horizontal and vertical pan; and rotate an image displayed on a monitor. Voice utterance signals may be decoded into at least five of those categories. Voice utterance signals may be decoded into at least three of those categories. The voice utterances may control water or gas insufflation via a voice utterance directed to distension or contraction of a surgical cavity. The issued control commands may be directed to increasing or decreasing of flow of insufflation fluid. The speech recognition technology may be trained to a single person's voice. The speech recognition technology may filter out sounds other than that single person's voice. An endoscope may have a microphone mounted therein. An endoscope may have a microphone and a pushbutton mounted adjoining each other. A database may provide data to be used by the speech recognition technology to improve decoding of the voice utterance signals. Data from the database may be obtained based at least in part based on an indication of identification of a specific endoscope. The identification of the identified endoscope may be obtained via a machine read of a machine-readable identification code of the identified endoscope. The database may further store operational properties of individual endoscopes. The processor may receive image data from an image sensor of the identified endoscope;

Various processes described herein may be implemented by appropriately programmed general purpose computers, special purpose computers, and computing devices. Typically a processor (e.g., one or more microprocessors, one or more microcontrollers, one or more digital signal processors) will receive instructions (e.g., from a memory or like device), and execute those instructions, thereby performing one or more processes defined by those instructions. Instructions may be embodied in one or more computer programs, one or more scripts, or in other forms. The processing may be performed on one or more microprocessors, central processing units (CPUs), computing devices, microcontrollers, digital signal processors, graphics processing units (GPUs), field programmable gate arrays (FPGAs), or like devices or any combination thereof. Programs that implement the processing, and the data operated on, may be stored and transmitted using a variety of media. In some cases, hard-wired circuitry or custom hardware may be used in place of, or in combination with, some or all of the software instructions that can implement the processes. Algorithms other than those described may be used.

Programs and data may be stored in various media appropriate to the purpose, or a combination of heterogeneous media that may be read and/or written by a computer, a processor or a like device. The media may include non-volatile media, volatile media, optical or magnetic media, dynamic random access memory (DRAM), static ram, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, other non-volatile memories, any other memory chip or cartridge or other memory technologies.

Databases may be implemented using database management systems or ad hoc memory organization schemes. Alternative database structures to those described may be readily employed. Databases may be stored locally or remotely from a device which accesses data in such a database.

In some cases, the processing may be performed in a network environment including a computer that is in communication (e.g., via a communications network) with one or more devices. The computer may communicate with the devices directly or indirectly, via any wired or wireless medium (e.g. the Internet, LAN, WAN or Ethernet, Token Ring, a telephone line, a cable line, a radio channel, an optical communications line, commercial on-line service providers, bulletin board systems, a satellite communications link, a combination of any of the above). Transmission media include coaxial cables, copper wire and fiber optics 430, including the wires that comprise a system bus coupled to the processor. Transmission may occur over transmission media, or over electromagnetic waves, such as via infrared, Wi-Fi, Bluetooth, and the like, at various frequencies using various protocols. Each of the devices may themselves comprise computers or other computing devices, such as those based on the Intel® Pentium® or Centrino™ processor, that are adapted to communicate with the computer. Any number and type of devices may be in communication with the computer.

A server computer or centralized authority may or may not be necessary or desirable. In various cases, the network may or may not include a central authority device. Various processing functions may be performed on a central authority server, one of several distributed servers, or other distributed devices.

The following applications are incorporated by reference. U.S. Provisional application Ser. No. 63/770,259, filed Mar. 11, 2025, titled Endoscope with Voice Activation; U.S. Provisional Application Ser. No. 63/739,458, filed Dec. 27, 2024, titled Endoscope with Improved Fluid Flow; U.S. application Ser. No. 18/796,226, filed Aug. 6, 2024, titled Endoscope; U.S. Provisional Application Ser. No. 63/666,540, filed Jul. 1, 2024, titled Endoscope; U.S. Provisional application Ser. No. 63/666,540, filed Jul. 1, 2024, titled Endoscope; U.S. Provisional Application Ser. No. 63/662,337, filed Jun. 20, 2024, titled Endoscope; U.S. Provisional Application Ser. No. 63/570,678, filed Mar. 27, 2024, titled Endoscope with Voice Activation; U.S. application Ser. No. 18/403,439, filed Jan. 3, 2024; U.S. Provisional Application Ser. No. 63/544,608, filed Oct. 17, 2023, titled Artificial Intelligence Agent for Preparing Physician's Note; U.S. application Ser. No. 18/370,375, filed Sep. 19, 2023, titled Image Enhancement for Endoscope; U.S. Provisional application Ser. No. 63/538,485, filed Sep. 14, 2023, titled Endoscope; U.S. Provisional application Ser. No. 63/534,855, filed Aug. 27, 2023, titled Endoscope; U.S. Provisional application Ser. No. 63/531,239, filed Aug. 7, 2023, titled Endoscope; U.S. Provisional application Ser. No. 63/437,115, filed Jan. 4, 2023, titled Endoscope with Identification and Configuration Information; U.S. application Ser. No. 17/954,893, filed Sep. 28, 2022, titled Illumination for Endoscope; U.S. Provisional Application Ser. No. 63/376,432, filed Sep. 20, 2022, titled Super Resolution for Endoscope Visualization; U.S. application Ser. No. 17/896,770, filed Aug. 26, 2022, titled Endoscope; U.S. Provisional Application Ser. No. 63/400,961, filed Aug. 25, 2022, titled Endoscope; U.S. application Ser. No. 17/824,857, filed May 25, 2022, titled Endoscope; U.S. Provisional Application Ser. No. 63/249,479, filed Sep. 28, 2021, titled Endoscope; U.S. Provisional Application Ser. No. 63/237,906, filed Aug. 27, 2021, titled Endoscope; U.S. application Ser. No. 17/361,711, filed Jun. 29, 2021, titled Endoscope with Bendable Camera Shaft; U.S. Provisional Application Ser. No. 63/214,296, filed Jun. 24, 2021, titled Endoscope with Bendable Camera Shaft; U.S. Provisional Application Ser. No. 63/193,387 titled Anti-adhesive Window or Lens for Endoscope Tip; U.S. Provisional Application Ser. No. 63/067,781, filed Aug. 19, 2020, titled Endoscope with Articulated Camera Shaft; U.S. Provisional Application Ser. No. 63/047,588, filed Jul. 2, 2020, titled Endoscope with Articulated Camera Shaft; U.S. Provisional Application Ser. No. 63/046,665, filed Jun. 30, 2020, titled Endoscope with Articulated Camera Shaft; U.S. application Ser. No. 16/434,766, filed Jun. 7, 2019, titled Endoscope with Disposable Camera Shaft and Reusable Handle; U.S. Provisional Application Ser. No. 62/850,326, filed May 20, 2019, titled Endoscope with Disposable Camera Shaft; U.S. application Ser. No. 16/069,220, filed Oct. 24, 2018, titled Anti-Fouling Endoscopes and Uses Thereof; U.S. Provisional Application Ser. No. 62/722,150, filed Aug. 23, 2018, titled Endoscope with Disposable Camera Shaft; U.S. Provisional Application Ser. No. 62/682,585 filed Jun. 8, 2018, titled Endoscope with Disposable Camera Shaft.

For clarity of explanation, the above description has focused on a representative sample of all possible embodiments, a sample that teaches the principles of the invention and conveys the best mode contemplated for carrying it out. The invention is not limited to the described embodiments. Well known features may not have been described in detail to avoid unnecessarily obscuring the principles relevant to the claimed invention. Throughout this application and its associated file history, when the term “invention” is used, it refers to the entire collection of ideas and principles described; in contrast, the formal definition of the exclusive protected property right is set forth in the claims, which exclusively control. The description has not attempted to exhaustively enumerate all possible variations. Other undescribed variations or modifications may be possible. Where multiple alternative embodiments are described, in many cases it will be possible to combine elements of different embodiments, or to combine elements of the embodiments described here with other modifications or variations that are not expressly described. A list of items does not imply that any or all of the items are mutually exclusive, nor that any or all of the items are comprehensive of any category, unless expressly specified otherwise. In many cases, one feature or group of features may be used separately from the entire apparatus or methods described. Many of those undescribed alternatives, variations, modifications, and equivalents are within the literal scope of the following claims, and others are equivalent. The claims may be practiced without some or all of the specific details described in the specification. In many cases, method steps described in this specification can be performed in different orders than that presented in this specification, or in parallel rather than sequentially.

The invention claimed is:

1. Apparatus, comprising:
a processor;
a machine readable nontransitory memory, having stored therein, instructions that, when executed, cause the processor to:
to obtain data from a database designed to store properties specific to speech of a specific surgeon scheduled to used an identified endoscope, and/or information indicating spoken utterances associated with a specific procedure for which the identified endoscope is to be used;
to accept signals of voice utterances from a surgeon using an endoscope;
to use speech recognition technology to decode the voice utterance signals into commands to control the endoscope, the decoding to be based at least in part on the properties specific to speech of the specific surgeon, and/or information indicating spoken utterances associated with the specific procedure, and to issue control commands to components of the endoscope to implement the decoded voice utterances;
concurrently with the accepting and decoding of voice utterances, to accept and decode control signals from another input device, and to issue control commands to components of the endoscope to implement the decoded other input control signals;
to accept the voice utterances and other input control signals concurrently and to issue the control commands to the components concurrently.

2. Apparatus, comprising:
a processor;
a machine readable nontransitory memory, having stored therein, instructions that, when executed, cause the processor to:
to accept signals of voice utterances from a surgeon using an endoscope, to use speech recognition technology to decode the voice utterance signals into commands to 6 control the endoscope, and to issue control commands to components of the endoscope to implement the decoded voice utterances;
concurrently with the accepting and decoding of voice utterances, to accept and decode control signals from another input device, and to issue control commands to components of the endoscope to implement the decoded other input control signals;
to accept the voice utterances and other input control signals concurrently and to issue the control commands to the components concurrently.

3. The apparatus of claim 2, the instructions further programmed to cause the processor to:
to obtain data from a database storing properties specific to speech of, or voice recognition training data specific to, a specific surgeon scheduled to used an identified endoscope, and/or information indicating spoken utterances associated with a surgical procedure for which the identified endoscope is to be used;
to use speech recognition decoding to be based at least in part on the data obtained from the database.

4. The apparatus of claim 2, the instructions further programmed to cause the processor to:
accept and decode voice utterance signals and control signals from two other input devices, concurrently, and to issue control commands to implement the decoded signals.

5. The apparatus of claim 4, the instructions further programmed to cause the processor to:
accept and decode control signals from at least two of a pushbutton on the endoscope, a front panel of a control unit of the endoscope, and a foot pedal.

6. The apparatus of claim 2, the instructions further programmed to:
accept and decode voice utterance signals and control signals from the other input device, the decoded signals yielding the same command to the same component of the endoscope.

7. The apparatus of claim 2, the instructions further programmed to:
cause the processor to decode the voice utterance signals into control commands from at least four categories from the group of categories consisting of:
start and stop recording of video;
raise and lower illumination;
control water or gas insufflation;
adjust image imaging parameters including at least two of contrast, saturation, color temperature, and white balance;
increase or decrease zoom;
change focal distance;
horizontal and vertical pan; and
rotate an image displayed on a monitor.

8. The apparatus of claim 2, the instructions further programmed:
to control water or gas insufflation via a voice utterance directed to distension or contraction of a surgical cavity, the issued control commands directed to increasing or decreasing of flow of insufflation fluid.

9. The apparatus of claim 2, the instructions further programmed to cause the processor to:
train the speech recognition technology to a single person's voice and to filter out sounds other than that single person's voice.

10. The apparatus of claim 2, further comprising:
an endoscope with a microphone mounted therein.

11. The apparatus of claim 10, wherein:
the endoscope has the microphone and a pushbutton mounted adjoining each other.

12. The apparatus of claim 2, the instructions further programmed:
to obtain data from the database at the computer, data to be used by the speech recognition technology to improve decoding of the voice utterance signals.

13. Apparatus, comprising:
a processor;
a machine readable nontransitory memory, having stored therein, instructions that, when executed, cause the processor to:
to obtain data from a database storing properties specific to speech of, or voice recognition training data specific to, a specific surgeon scheduled to use an identified endoscope, and/or information indicating spoken utterances associated with a surgical procedure for which the identified endoscope is to be used;
to accept signals of voice utterances from a surgeon using the identified endoscope;
to use speech recognition technology to decode the voice utterance signals into commands to control the endoscope, the decoding to be based at least in part based at least in part on the data obtained from the database, and to issue control commands to components of the endoscope to implement the decoded voice utterances.

14. The apparatus of claim 13, the instructions further programmed to cause the processor to:
concurrently, accept and decode control signals from another input device, and to issue control commands to components of the endoscope to implement the decoded other input control signals;
to accept the voice utterances and other input control signals concurrently and to issue the control commands to the components concurrently.

15. The apparatus of claim 13, the instructions further programmed to cause the processor to:
to obtain the data from the database based at least in part based on an indication of identification of a specific endoscope.

16. The apparatus of claim 15, the instructions further programmed to cause the processor to:
to obtain the identification of the identified endoscope via a machine read of a machine-readable identification code of the identified endoscope.

17. The apparatus of claim 15, wherein:
the database further stores operational properties of individual endoscopes.

18. The apparatus of claim 15, the instructions further programmed to:
to receive image data from an image sensor of the identified endoscope;

19. The apparatus of claim 13, the instructions further programmed to:
cause the processor to decode the voice utterance signals into control commands from at least four categories from among the group of categories consisting of:
start and stop recording of video;
raise and lower illumination;
control water or gas insufflation;
adjust image imaging parameters including at least two of contrast, saturation, color temperature, and white balance;
increase or decrease zoom;
change focal distance;
horizontal and vertical pan; and
rotate an image displayed on a monitor.

20. The apparatus of claim 13, further comprising:
an endoscope with a microphone mounted therein.

21. The apparatus of claim 20, wherein:
the endoscope has the microphone and a pushbutton mounted adjoining each other.
